(19) United States
(12) Patent Application Publication
(10) Pub. No.: US 2026/0276532 A1
(43) Pub. Date: Sep. 17, 2026

(54) DEVICE FOR THE NON-DESTRUCTIVE DETERMINATION OF PLANT VITALITY PARAMETERS, AND, IF APPLICABLE, THE AUTOMATED TREATMENT OF DEFICIENCY CONDITIONS AND DISEASE CONDITIONS, AND METHOD THEREFOR

(71) Applicant: RHENAC GreenTec AG, Hennef (DE)

(72) Inventor: Horst THEISEN, Bonn (DE)

(73) Assignee: RHENAC GreenTec AG, Hennef (DE)

(21) Appl. No.: 19/669,534

(22) Filed: May 6, 2026

Related U.S. Application Data
(63) Continuation of application No. PCT/EP2024/081349, filed on Nov. 6, 2024.

(30) Foreign Application Priority Data
Nov. 6, 2023 (DE) 10 2023 130 600.6

(51) Int. Cl. G01N 21/31; G01N 33/00

(52) U.S. Cl. CPC G01N 21/31; G01N 33/0098

(57) ABSTRACT

A device for the non-destructive determination of vitality parameters of plants comprises: a lighting unit; at least three photocells that separately quantitatively determine white, blue and red light; a unit for evaluating the light measured by the photocell; a database with stored values related to plant vitality; and a data processing system which compares the measured values with the stored values (indices), wherein the lighting unit and the photocells are positioned at a distance from the plant parts to be determined so that they do not touch them, and are surrounded by a light-impermeable hood (cover) which encloses the plant parts, the lighting unit, and the photocells.

This nonprovisional application is a continuation of International Application No. PCT/EP2024/081349, which was filed on Nov. 6, 2024, and which claims priority to German Patent Application No. 10 2023 130 600.6, which was filed in Germany on Nov. 6, 2023, and which are both herein incorporated by reference.

### BACKGROUND OF THE INVENTION

### Field of the Invention

The present invention relates to a device for the non-destructive determination of plant conditions by means of vitality parameters, as well as a method therefor. It also applies, if applicable, to the automated treatment of deficiency conditions and/or disease conditions of such plants.

### Description of the Background Art

Plant sensors are well known. Reflection patterns generated by sunlight or an artificial light source are determined and insights are obtained from this for targeted thinning.

Optimization of plant growth by irradiation with suitable light sources is well known. For example, DE 102019110989 A1, which corresponds to US 2022/0204897, which is incorporated herein by reference, describes a light and temperature device that can be used to optimize the production of biomass in bioreactors. In this process, an LED light arrangement with LED light arrays and cooling fins is arranged on a translucent incubation container. The LEDs are directed towards the incubation container, and the cooling fins point away from the LED light. The distance between the light arrangement and the incubation container should be as small as possible.

EP 3 389 357 B1, which is incorporated herein by reference, also describes a device for the illumination of large turfs, wherein lighting elements are mounted on a carrier that is positioned above the turf and moves across the area being treated. This stimulates growth throughout the entire turf, especially at sports facilities.

DE 10 2010 034 603 B4, which corresponds to US 2013/0152464, describes a sensor system for determining an optical property, in particular the chlorophyll content or other disease or pest infestation of a plant or parts thereof, by measuring the reflection of incident light. By means of a first light source, light is emitted along a first beam axis and by means of a second light source, light along a second beam axis, and by means of at least one receiver along a third beam axis to capture the light reflected by the plant. By means of this device, it is possible to determine the chlorophyll content of a plant, but it is not possible to infer a specific condition, e.g., the disease condition of the plant, and then counteract it in a targeted manner.

A device for detecting the fertilization condition of plants is known from WO 2020/253915 A, which includes a light source, a receiver, a time recorder and an evaluation unit. The light source sends a light signal for a predefined period, which is received by a receiver after this period. A quotient between the minimum and maximum signal is determined and compared with a threshold value. In this way, the fertilization status of the plants is then determined.

However, many of the conventional methods have proven to be very inaccurate and only result in an analysis of the respective plant condition that is fraught with errors.

### SUMMARY OF THE INVENTION

It is therefore an object of the invention to provide a device and a method for improved detection of plant vegetation conditions. In addition, components are to be provided with which these conditions can be remedied in a targeted manner even without the external addition of fertilizers and plant nutrients.

According to an example of the invention, the method involves irradiating plants with precisely selected light of specific wavelengths, and determining its reflection, absorption and, where applicable, fluorescence, and deriving an index from the ratio of the intensities of the different wavelengths, which is then compared with a set of indices stored in a database. The indices stored in the database have previously been determined for all possible deficiency conditions of the plants such as stress, drought, cold, heat, lack of light, etc. By comparison with the values, data or indices stored in the database, the vegetation condition of the plants is then determined. This method makes it possible to non-destructively determine the composition of plant compounds and to reduce or completely eliminate undesirable values such as deficiencies or stress hormones in the plants.

If a vegetation condition is known, it can be improved or completely eliminated, for example, by appropriate irradiation with selected light of certain wavelengths. With the help of appropriate wavelengths, the plant can now be stimulated to produce or synthesize the substances necessary to correct the deficiency. In this way, it is possible to stimulate the plant to synthesize the substances necessary to combat the deficiency condition without the external addition of fertilizer or other aids.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 illustrates a ratio of various wavelength ranges; and

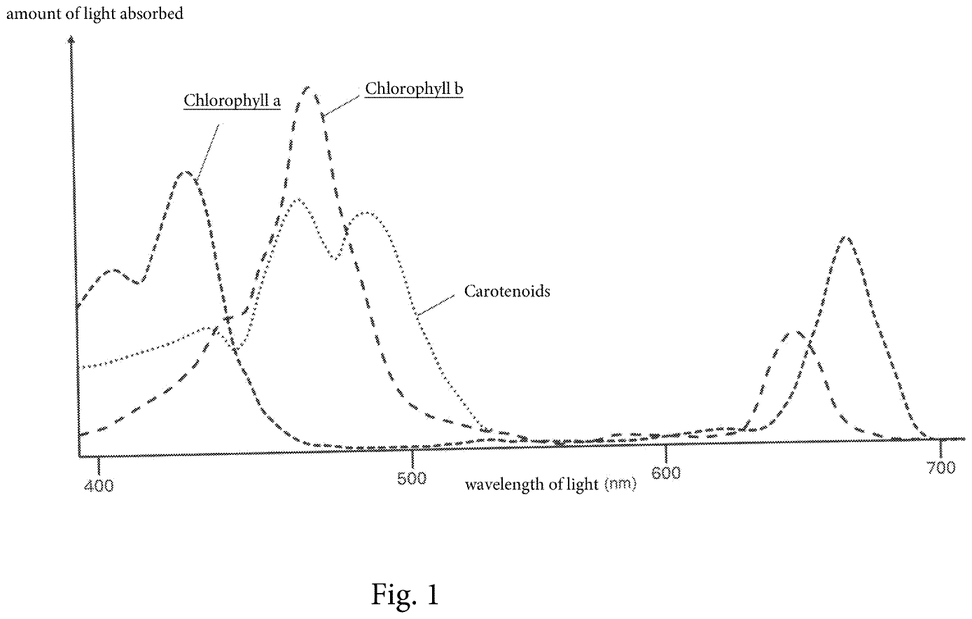

FIG. 2 illustrates an example of the device of the invention.

### DETAILED DESCRIPTION

The invention is based on the knowledge that, for example, wavelength ranges emitted or reflected by the plant in the blue range of 400 nm, preferably from 410 nm to typically 470 nm, preferably up to 460 nm—especially up to 450 nm-—allow for insights into the chlorophyll content of the plant.

Corresponding measured values in the green/yellow range of 500 nm, especially from 510 nm to 580 nm and especially up to 570 nm and preferably up to 560 nm, indicate stress in the plant. This is caused, for example, by the anthocyanin content. In contrast, measured reflected values in the wavelength range of 570 nm, particularly 580 nm, especially up to 645 nm—preferably up to 645 nm—indicate the presence of dead material, with a range from 585 nm to 670 nm having proven to be particularly suitable.

Measured values in the range from 670 nm to 770 nm, preferably up to 760 nm, indicate in particular the photosynthesis rate of the respective plant.

Corresponding values in the dark red range, starting from 800 nm, preferably between 810 nm and 930 nm, especially up to 920 nm-preferably up to 900 nm-give indications of the water balance of the plant.

A range from 680 nm to 780 nm in particular has proven to be particularly suitable. In this way, it is possible to determine the respective plant condition by comparing the respective intensities of the measured wavelengths to each other. These values are then compared with values or indices stored in a database and previously precisely determined, thus making it possible to determine the condition of the respective plants.

In this case, it has proven to be particularly useful if the measurements are carried out with the device according to the invention under a light-impermeable cover or hood. It is preferable to position the lighting and measuring elements at a distance from the plant, i.e., without direct contact with the plant itself. It has been shown that particularly reliable values are obtained in this way. Preferred distances are at least 5 cm, preferably at least 10 cm, wherein a minimum distance of 15 cm, particularly at least 18 cm, is preferred. Standard spacing is typically up to 2 meters, though 1 meter or less—and preferably 50 cm or less—is preferred. Distances of 40 cm or less, especially 30 cm or 25 cm, have proven to be particularly suitable. The entire measuring system, which is enclosed by the hood or cover, is placed directly on the ground, and the plant or plurality of plants or a plant community to be examined is measured without the influence of external light. For the hood or cover, diameters of at least 3 cm, especially at least 4 cm or 5 cm, have proven to be useful. Maximum diameters are 50 cm, especially 40 cm, with 30 or 20 cm being preferred. Here, too, diameters of up to 10 cm have proven to be useful.

The shapes of the hood or cover can be arbitrarily designed, as long as they sufficiently exclude external light. This means that these can be shaped like a bell, or a round or square cylinder. The shape of a pyramid is also a suitable bell shape. In another expedient arrangement, the inside of the bell shape/cover is reflective, which can be achieved, for example, with a bright inner surface. A mirrored inner surface has also proven to be useful. It has been shown that even more accurate readings are obtained in this way.

Reflectance or spectral measurement is based on the partial reflection of the light incident on the plant or parts of it, which can be measured. The incident light is partially absorbed by the plant pigments of the leaves or other parts. One of the most important absorbers is chlorophyll, which is responsible for photosynthesis with the support of carotenoids and absorbs mainly in the blue and red spectral range (see the figure).

Other plant pigments, such as anthocyanins or flavonols, also absorb certain parts of the spectrum to protect the plant from external influences.

However, a certain proportion of the incident radiation is always reflected. The ratio of reflection in certain areas of the visible (VIS) and near-infrared (NIR) range can also be used for the non-destructive analysis of phytonutrients. Since these indicate the respective vegetation condition of the plant, these values can be determined. For this purpose, many spectra and wavelengths—particularly those most relevant to various vegetation conditions, such as heat, drought, frost, stress caused by pruning wounds, etc. —have been identified and stored in a database against which current measurements are compared.

By using a bell shape or a cover, the accuracy of the plant radiation analysis can be increased to well over 90%, especially to at least 92% or 94%. A device for the non-destructive determination and modification of the plant vitality parameters comprises a lighting unit as well as at least three photocells, each of which separately quantitatively detects and determines white, blue and red light, as well as a unit for evaluating the light intensities measured by the photocell(s), which includes a database containing indices of plant vitality previously determined for various conditions. In addition, the device contains a data processing system which compares the measured values or parameters with the stored values and thus determines the plant condition. In this way, the necessary wavelengths are identified that stimulate the plant to synthesize vegetative substances that are required to correct the identified deficiencies.

A number of LED sensors have proven to be suitable for use as lighting units with a photocell. Such LED arrangements are commercially available and combined into a single spectroscopy sensor, for example by the company Sparkfun. Such sensors preferably have several measuring ranges or channels. Typical sensors comprise at least 5, preferably at least 10 measurement channels, wherein at least 13 or 14 measurement channels are preferred. There is no upper limit for the number of measurement channels. This is determined only by the size of the device or by the size of the sensors. For example, a typical number of measurement channels is between 15 and 20, with 17 to 19 and especially 18 measurement channels proving to be appropriate. As described above, such sensor boards are arranged in a light-impermeable casing or a cover (light measuring bell), the shape of this cover (bell shape or protective cap) can be arbitrarily designed. The only requirement is that it must be large enough to accommodate the sensor and that it covers the plants to be identified. Typically, the casing/bell shape is dark on its outside, in particular black.

The sensor is installed in a casing or bell-shaped cap with a length of at least 8 cm, which is intended to shield natural light. In the case of sports turf, a distance of 2.5-3.0 cm between the sensors and the turf is usually chosen, which means that the distance from the sensor to the turf is at least approx. 5 cm.

This is to prevent individual plant parts, such as blades of grass, from influencing the measurement result too much and to create a suitable average value of the measured plants in this area.

For a preferred approach according to the invention, measured values or indices of plant physiology are first determined (main indices). Also, three secondary indices for the plant conditions are determined. This effectively results in four main indices (plant physiology): plant density, chlorophyll content, anthocyanin content and flavonol content and three secondary indices (plant condition): (plant health, water status and photosynthetic activity).

When measuring with the light sensor, the aim is to measure the reflection of the light through the plant. Since the reflection changes with respect to the light irradiation, a constant exposure system that is not subject to fluctuations is preferred. With an optical sensor and built-in UV LED 395-405 nm, IR 730-745 nm, blue 430-445 nm, red 660 nm and white 4,000-5,000 Kelvin LED (all the above figures can deviate by +/−25%, preferably by ±20%, in particular by ±10%) as the light source in a light measuring bell, natural daylight is eliminated from the measurement as best as possible. The individual LEDs are distributed over at least 18-24 measurement channels, and the corresponding wavelengths are then assigned to the channels in such a way that the measured wavelengths are evaluated via the channels. The wavelengths can be easily evaluated with previously determined values from the database. It has been shown that the wavelengths of values 395-410-435-450-510-535-560-610-645-680-705-730-760-810-860-900-940 nm are particularly suitable (each can deviate by ±5%, in particular 2%). Each wave peak has its own sensitivity to light.

A large number of measurements were carried out under different light conditions and, at different times during the calendar year, a wide variety of reference measurements were made and compiled into a database. Based on this data, a coefficient was then determined for each measurement channel (wavelength) which can be used to calibrate the measured value and interpret it correctly from a metrological standpoint. The measurements revealed that the measured reflection in the individual channels depends significantly on the plant's compounds. These measured values provide insights into the condition of the plant being measured.

The absorption spectra of chlorophyll and anthocyanin were used for further calculations. The ratio of various wavelength ranges was determined in order to ascertain the respective plant density and the chlorophyll and anthocyanin content (see the figure).

Based on this, indices for plant conditions and their phytohormones can now be built and stored in a database. The database can be expanded as desired by further measurements.

While the values in the blue component of approx. 410-450 nm indicate reliable insights into the chlorophyll content and the values in the green/yellow range of approx. 510-560 nm indicate reliable values for stress in the plant (anthocyanin content), the range of approx. 585-645 nm serves as an indicator for dead material (death markers). Values in the range of approx. 680-760 nm enable insights into the health and especially the photosynthetic rate of the measured plant. Values in the dark red range of approx. 810-900 nm allow for an assessment of the plant's water balance.

The measurement channels are also suitable for determining further indices and forwarding them to the data processing department, thus enabling a continuous process of further developments of the existing plant measurement system.

In the context of the present invention, the previously available measured values were determined under the exclusion of natural light and these data were prepared for digitization and thus are used for further processing by being incorporated into control loops (additional lighting, turf heating, air cooling, automatic fertilizer application, and assessment of plant damage caused by maintenance equipment (stress on the plant caused by dull blades in the mower), targeted irrigation, preventive measures to protect against turf diseases) and much more. When using mobile turf management systems, it is possible to continuously generate turf data, which are then stored in a cloud as a digital turf twin (digital representation of the turf condition) for further processing and use by other devices (GPS coordinates as measuring points and data sets for other applications). In this way, an image is generated from measuring points in which the pixels represent the size of the respective individual measurement.

The device according to the invention can be configured both as a handheld device and as part of a mobile, in particular self-propelled unit (similar to a lawn mower robot). Preferably, such a device according to the invention also has a positioning device such as a GPS device. In this way, the data sets determined by the device can be transmitted, preferably wirelessly, to a central system comprising the database and the data processing system.

Plants suitable for the device according to the invention typically grow in large areas, such as sports facilities, particularly sports turf. However, it is also possible to assess the condition of other large-scale systems, such as fields, and then alter the condition of these plants through appropriate light treatment. This is particularly suitable for the increasingly common food production systems using artificial light in enclosed spaces, such as those necessary to secure global food supplies.

However, the invention also relates to a method for the non-destructive determination of the vegetation condition of plants, wherein the plants are exposed accordingly and the reflected and/or absorbed and/or generated fluorescent light is examined using photocells. Each photocell measures at least one defined wavelength. By comparing the wavelength intensities to each other, indices can be generated that are compared with stored indices of a database containing various plant vegetation conditions, and thus, the vegetation condition of the plants measured here can be determined. In this way, it is possible to correct corresponding deficiencies by irradiating the plants with the respective wavelengths. The invention therefore also relates to a method and a lighting system for correcting plant deficiencies through irradiation with light at wavelengths or wavelength ranges that stimulate or promote the synthesis of the plant substances necessary to correct such deficiencies, such as phytohormones, etc.

Any known photo sensors are suitable as photo sensors, wherein photo sensors such as those known from digital cameras have proven to be suitable here. Typical examples of such photosensors are CCD sensors or CMOS sensors. In principle, BSI-CEMOS sensors are also suitable.

As light sources for irradiation, LED lights have been, or are, preferably suitable. Such LED lights are also commercially available for any wavelength. In principle, it is also possible to use specific wavelengths by placing filters in front of the light source.

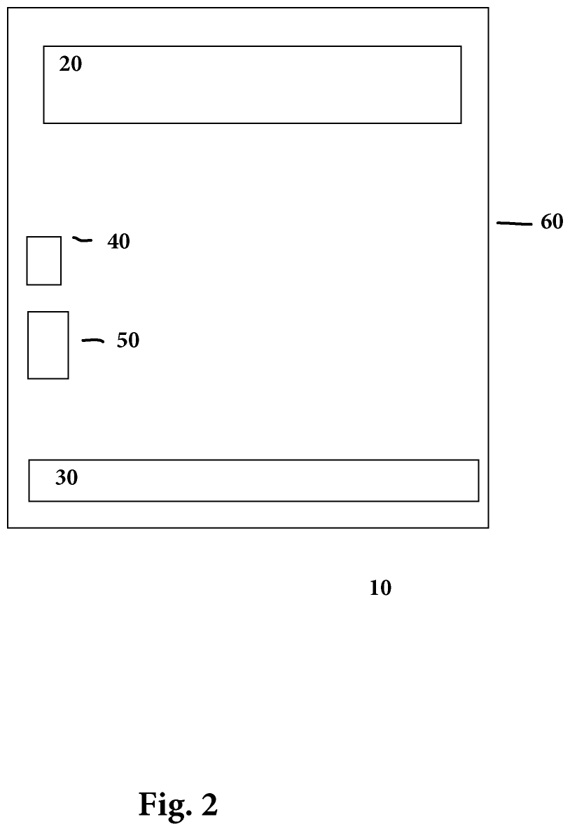

FIG. 2 illustrates a device 10 for a non-destructive optical determination of plant vitality conditions. the device comprises a lighting unit 20 with at least one wavelength selected from white, blue and red light, as well as a measuring unit 30 having at least three photocells, each of which quantifies separately white, blue and red light; an evaluator for evaluating the light measured by the photocells. A database 40 has stored values related to plant vitality (vegetation condition) and a data processing system 50 compares the measured values with the stored values (indices). The lighting unit 10 is positioned at a distance from the plant parts to be measured so that the lighting unit does not touch the plant parts, and wherein the plants, lighting unit and measuring unit are surrounded by a light-impermeable cover 60 in such a way that external light is eliminated.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A method for a non-destructive optical determination of vegetation conditions of plants, the method comprising:
illuminating the plants with a lighting unit;
selecting at least one wavelength from white, blue or red light;
determining an intensity of the reflected and/or absorbed light and/or generated fluorescent light via measuring elements having at least three photocells, which separately and quantitatively determine white, blue and red light, and each of which detects at least one defined wavelength;
forming indices from the ratios of the intensities of different wavelengths to one another;
comparing the indices obtained with the index values stored in a database for various plant vegetation conditions; and
determining the vegetation condition using the values of deficiencies and/or stress conditions stored in the database,
wherein the lighting unit and measuring element are distanced from the plant surface,
wherein the lighting unit and measuring element are surrounded by a light-impermeable cover which sufficiently excludes external light and which also surrounds the plants to be analyzed, such that the intensity of the reflected and/or absorbed light and/or fluorescent light produced is determined without the influence of external light, and
wherein the values/indices measured by the lighting element and measuring element are digitally transmitted to a database.

2. The method according to claim 1, wherein the plants form a lawn, in particular a sports turf.

3. The method according to claim 1, wherein at least 10 or 15 wavelengths are acquired and transmitted in the measurement channels to the data processing system and database in digital form.

4. The method according to claim 1, wherein the distance from the plant is at least 5 cm.

5. The method according to claim 1, wherein the measuring element comprises photocells.

6. The method according to claim 1, wherein the content of chlorophyll, carotenoids, anthocyanins, and/or flavonols are determined via the measured light intensities.

7. The method according to claim 1, wherein the lighting system produces light in the UV range of 395 nm to 405 nm, in the range of 730 nm to 745 nm, blue in the range of 430 nm to 445 nm, red in the range of 650 nm to 670 nm and white in the range of 4000 to 5000 Kelvin.

8. The method according to claim 1, wherein the undesirable plant conditions are corrected by supplementary lighting, turf heating or soil heating, air cooling, automatic fertilization, assessment of damage caused by maintenance equipment such as lawn mowers, targeted irrigation, or preventive measures to protect against plant diseases.

9. The method according to claim 8, wherein the plants are exposed to light sources and photocells at wavelengths that stimulate synthesis of deficient plant substances in order to correct the deficiencies.

10. A device for a non-destructive optical determination of plant vitality conditions, the device comprising:
a lighting unit with at least one wavelength selected from white, blue and red light, as well as a measuring unit having at least three photocells, each of which quantifies separately white, blue and red light;
an evaluator for evaluating the light measured by the photocells;
a database with stored values related to plant vitality (vegetation condition); and
a data processing system that compares the measured values with the stored values (indices),
wherein the lighting unit is positioned at a distance from the plant parts to be determined so that the lighting unit does not touch the plant parts, and
wherein the plants, lighting unit and measuring unit are surrounded by a light-impermeable cover in such a way that external light is eliminated.

11. The device according to claim 10, wherein the cover is black on an outside.

12. The device according to claim 10, wherein the lighting unit is controllable in such a way that it selectively generates only previously determined wavelengths and wavelength ranges in order to selectively stimulate plants to form desired synthesis products.

13. The device according to claim 10, wherein it is positioned on a self-propelled mobile unit.

14. The device according to claim 13, further comprising a positioning device and/or GPS.
